(19) United States
(12) Patent Application Publication
(10) Pub. No.: US 2024/0099769 A1
(43) Pub. Date: Mar. 28, 2024

(54) Methods and Systems for Thermal Enhancement of Electroporation

(71) Applicant: Virender K. Sharma, Paradise Valley, AZ (US)

(72) Inventor: Virender K. Sharma, Paradise Valley, AZ (US)

(21) Appl. No.: 18/477,449

(22) Filed: Sep. 28, 2023

Related U.S. Application Data
(60) Provisional application No. 63/377,496, filed on Sep. 28, 2022.

(51) Int. Cl. A61B 18/14

(52) U.S. Cl. CPC A61B 18/1492; A61B 2018/00577

(57) ABSTRACT

System and methods for enhancement of nonthermal electroporation using thermal ablation include a system having a catheter for providing concomitant thermal energy and electroporation and a controller for controlling the concomitant delivery of the thermal energy and electroporation. The method includes controlled application of thermal energy in conjunction with electroporation to increase a region of irreversible electroporation.

### CROSS-REFERENCE

The present application relies on U.S. Patent Provisional Application No. 63/377,496, titled “Methods and Systems for Thermal Enhancement of Nonthermal Irreversible Electroporation”, and filed on Sep. 28, 2022, for priority. The above-mentioned application is herein incorporated by reference in its entirety.

### FIELD

Embodiments of the present specification relate to methods and systems configured to deliver vapor for thermal ablation in combination with nonthermal electroporation.

### BACKGROUND

Electroporation is a biophysical phenomenon that was discovered in the 1960s. In the 1960s it was known that by applying an external electric field, a large membrane potential at the two poles of a cell can be created. In the 1970s it was discovered that when a membrane potential reached a critical level, the membrane would break down and that it could recover. By the 1980s, this opening was being used to introduce various materials/molecules into the cells. Electroporation can be broadly divided into reversible electroporation (RE) and irreversible electroporation (IRE).

Irreversible electroporation (IRE) is based on the principle of electroporation or electropermeabilization, in which electric pulses or high-amplitude electric fields of sufficient duration are used to create nanoscale defects in the cell membrane. These defects, termed “nanopores” or “conductive pores”, permeate the cell membrane, permitting ions and molecules to pass in and out of the targeted cells. This phenomenon is manifested in two distinct forms: reversible electroporation (RE), in which permeabilizing structures are transient and membrane integrity is quickly recovered thereby not leading to cell death; and IRE, in which permeabilization disrupts cellular homeostasis and leads to cell death.

Although these nanopores can appear spontaneously, external electric fields lower the activation energy necessary for the stochastic pore formation process, resulting in the production of pores at a higher rate. When the electrical stimulus is of sufficient strength, water dipoles on either side of the bilayer reorient to the field and their interaction becomes favorable. Initially, the water column spanning the membrane is highly unstable, forming hydrophobic pores or “water wires”. As electrical energy is delivered to the system and water molecules penetrate the membrane, many of the initial structures evolve into long-lived hydrophilic pores. This transition is mediated by reorientation of the polar fatty acid head groups into a more energetically favorable alignment, thereby stabilizing the pore. Simulations predict that hydrophobic pores are <1 nm in diameter and reseal within milliseconds, whereas hydrophilic pores are roughly 1-10 nm in diameter and reseal within minutes to hours. External environmental influences such as thermal fluctuations or mechanical insult can enhance pore formation.

This ability of the cell membrane to reseal electropores, referred to as RE has been used for gene transfection (electrogenetherapy) and for introducing impermeable anticancer drugs in cells (electrochemotherapy). RE occurs when the electricity applied with the electrodes is below the electric field threshold of the target tissue (up to 1 kV). Because the electricity applied is below the cells' threshold, it allows the cells to repair their phospholipid bilayer and continue on with their normal cell functions. RE is typically done with treatments that involve getting a drug or gene (or other molecules that are not normally permeable to the cell membrane) into the cell. Not all tissue has the same electric field threshold; therefore, careful calculations need to be made prior to a treatment to ensure safety and efficacy. Stronger electric fields cause irreversible damage to the cell membrane, which cannot reseal the pores, resulting in cell death due to necrosis and/or apoptosis. This effect referred to as IRE, has been used in food processing technology. In 2005, IRE was proposed as a standalone soft tissue ablation technique. IRE is a predominantly nonthermal ablative technology that uses high-voltage, low-energy direct current (DC) pulses to induce cell death. IRE is a technology that uses DC current up to 3 kV to induce cell death.

Non-thermal irreversible electroporation (N-TIRE) has proven successful in treating many different types of tumors and other unwanted tissue. This procedure is done using small electrodes (about 1 mm in diameter), placed either inside or surrounding the target tissue to apply short, repetitive bursts of electricity at a predetermined voltage and frequency. These bursts of electricity increase the resting transmembrane potential (TMP), so that nanopores form in the plasma membrane. When the electricity applied to the tissue is above the electric field threshold of the target tissue, the cells become permanently permeable from the formation of nanopores. As a result, the cells are unable to repair the damage and die due to a loss of homeostasis. N-TIRE is unique to other tumor ablation techniques in that it does not create thermal damage to the tissue around it.

One major advantage of using N-TIRE is that, when done correctly according to careful calculations, it only affects the target tissue. Proteins, the extracellular matrix, and critical structures such as blood vessels and nerves are all unaffected and left healthy by this treatment. This allows for a quicker recovery and facilitates a more rapid replacement of dead tumor cells with healthy cells.

Before doing the procedure, scientists must calculate exactly what needs to be done and treat each patient on an individual case-by-case basis. To do this, imaging technology such as CT scans and MRIs are commonly used to create a 3D image of the tumor. From this information, they can approximate the volume of the tumor and decide on the best course of action including the insertion site of electrodes, the angle they are inserted in, the voltage needed, and more, using software technology. Often, a CT machine will be used to help with the placement of electrodes during the procedure, particularly when the electrodes are being used to treat tumors in the brain.

The entire procedure is very quick, typically taking about five minutes. The success rate of these procedures is high and is very promising for future treatment in humans. One disadvantage to using N-TIRE is that the electricity delivered from the electrodes can stimulate mu scle cells to contract, which could have lethal consequences depending on the situation. Therefore, a paralytic agent must be used when performing the procedure. The paralytic agents that have been used in such research are successful; however, there is always some risk, albeit slight, when using anesthetics.

The application of bipolar electroporation is highly controlled and can be customized to suit the specific requirements of the experiment or application. Parameters such as the pulse duration, amplitude, and frequency of electrical pulses can be adjusted to achieve optimal results. The pulse duration is typically in the range of microseconds to milliseconds, and the amplitude can range from tens to hundreds or even thousands of volts, depending on the cell type and experimental objectives. Bipolar electroporation limits or eliminates the inadvertent stimulation of muscle cells and hence the need for a paralytic agent.

Bipolar electroporation has several advantages over other methods of molecular delivery, such as viral transduction or chemical-based methods. First, it is a non-viral method, which eliminates the risk of insertional mutagenesis or immune response associated with viral vectors. Secondly, it allows for the efficient delivery of a wide range of molecules, including nucleic acids (DNA, RNA), proteins, drugs, and dyes. Thirdly, it can be applied to various types of cells, including cultured cells, primary cells, and even tissues in ex vivo or in vivo settings.

The transient nature of the nanopores formed during bipolar electroporation is a key feature of this technique. The pores spontaneously reseal shortly after the electrical pulse, restoring the integrity of the cell membrane. This reversibility ensures minimal damage to the cells and enables their survival and subsequent recovery.

Bipolar electroporation finds applications in various fields, including gene delivery, gene editing, drug delivery, and tissue engineering. In gene delivery, for example, foreign DNA or RNA molecules can be introduced into target cells to modify their genetic makeup or induce specific protein expression. This is particularly valuable in research studies aiming to understand gene function, disease mechanisms, or developing therapeutic interventions.

Overall, bipolar electroporation is a versatile and effective technique for introducing molecules into cells. Its ability to efficiently deliver a wide range of molecules without the limitations associated with viral or chemical-based methods makes it a valuable tool in biomedical research and clinical applications. Continued advancements in the field are expected to further enhance the precision and efficiency of bipolar electroporation, expanding its potential applications in the future.

High-frequency irreversible electroporation (H-FIRE) uses electrodes to apply bipolar bursts of electricity at a pulsatile voltage waveform with different characteristic frequencies between 250 kHz and 2 MHz, as opposed to unipolar bursts of electricity at a low frequency. FIG. 1 illustrates a comparison of a graph 102 showing DC pulse 104 applied in IRE and another graph 122 showing bipolar pulses 124 as used in H-FIRE. DC pulse 104 has a pulse width of approximately 100 μs (see x-axis 106 of graph 102), and a voltage of approximately 2500V (see y-axis 108 of graph 102). Subsequent DC pulses of similar duration and voltage as 106 and 108 are sequentially repeated after a delay 110 of approximately 1 second. Bipolar pulses 124 range from 2500V to −2500V (see y-axis 128 of graph 122). Each burst of bipolar pulses 124 last for a period of approximately 200 μs, where each pulse has a width 132 of approximately 1 μs. A delay 134 of approximately 1 μs between two consecutive pulses 133 marks the bursts of pulses 124. Further, a delay 130 of approximately 1 second is provided between two consecutive bursts 124. This type of procedure has the same tumor ablation success as N-TIRE. However, it has one distinct advantage that H-FIRE does not cause muscle contraction in the patient and therefore there is no need for a paralytic agent. Furthermore, H-FIRE voltage waveforms were shown to penetrate a heterogeneous system and have been demonstrated to produce more predicable ablations due to the lesser difference in the electrical properties of tissues at higher frequencies. H-FIRE also exhibits selectivity toward malignant phenotypes.

Typical electroporation generators use a capacitor size of 500 uF-6 mF, generate a peak voltage 1 kV-6 kV and a pulse width of 0.5 us-10 us. Supplying excessive electrical energy within a given time frame can not only cause thermal damage due to excessive heating but can also cause large area of IRE due to large electrical fields. Hence N-TIRE is utilized for soft tissue ablation by incorporating nonthermal parameters for specific tissues, by incorporating thermal mitigation strategies, or by modeling treatment beforehand to select pulse paradigms capable of N-TIRE. Typically, 50-100 pulses on the order of 100 μs in length are sequentially delivered between each electrode pair. The lethal threshold or irreversible threshold (Eᵢᵣ) is a metric of the susceptibility of a certain tissue or cell type to IRE induced cell death. It is dependent on the shape and amplitude of the characteristic waveform, number of pulses, and duration of the applied field, but for most tissues, this threshold is between 300 and 1000 V/cm when 100 pulses are applied. Voltage-to distance ratio (VDR, V/cm), defined as the quotient of the applied potential (V) and electrode separation (cm) is another important parameter of IRE field and energy. VDR for prostatic tissue is up to 3000 V/cm and spare the urethra, rectum, and capsule when probe is placed in close vicinity to this structure. Sexual function is also maintained. Target VDR for pancreatic tissue is 1500 V/cm and for liver tissue is 1000 V/cm.

It is critical to note that the electric field threshold for a given tissue decreases as more pulses are applied but saturates after a certain number of pulses. Moreover, because minor Joule heating effects occur during treatment, increased pulse numbers result in local increases in conductivity at a rate of 1-3%/° C., which can also propagate the electric field and increase ablation volume.

Additionally, the electrical stimulation parameters can be adjusted to create High frequency reversible electroporation (H-FRE) or nonthermal reversible electroporation (N-TRE).

Electroporation is believed to be a nonthermal phenomenon and seemingly affects only the cell membrane, while presumably leaving intact extracellular matrix. Although IRE is steadily gaining clinical acceptance, the very high energy delivered during tissue ablation (>3000 J) by application of a large sustained electric field (3 kV) raises the issue of chronic collateral thermal and non-thermal damage to other organs and acute proarrhythmic cardiac effects. Electroporation develops not only at sites with maximal field gradient but also at sites with maximal structural heterogeneity, because activating function is determined by both. Incidentally, atria have been shown to be more susceptible to electroporation than ventricles. With IRE, the distribution of current and electric field throughout the body due to application of very high energy could be expected to exacerbate the adverse effects. To mitigate acute cardiac safety concerns, IRE is sometimes gated with the R wave of the electrocardiogram (ECG) so that the electric pulse could be delivered during the absolute refractory period of the ventricles. IRE is synchronized with the absolute refractory period of the electrocardiogram (ECG) to mitigate the risk of electrical interference with cardiac myocytes and potential arrhythmia. Although ECG-gated IRE showed improved cardiac safety, there remains concerns about short- and long-term cardiac injury associated with R-gated IRE.

Research has indicated that even in absence of clinical manifestations of cardiac adverse events, subclinical injury can occur as measured by cardiac biomarkers and 15 patients (57%) exhibited procedure-related cardiac arrhythmias, suggesting a need for added safety measures. The application of an electric field during the IRE procedure affects not only tissue involved by cancer but also other organs on the path or near the fringes of the electric field, depending on the field configuration and intensity.

On the other hand, thermal ablation methods often result in non-uniform ablation of a target surface area. Optimal ablation (therapeutically effective ablation) is achieved in a small area of the target tissue but not throughout the target tissue. In other words, in one strip, optimal ablation may be achieved but extending out from the strip, suboptimal ablation is observed. In most cases, achieving above 50% ablation of the total target tissue after a single treatment is very hard. Increasing power (heat) or duration of the ablation to address the limitation of non-uniform ablation is likely to result in damaging adjacent healthy tissue due to an overdose.

Thermal ablative techniques such as radiofrequency energy, microwave, HIFU, and cryoablation provide a minimally invasive treatment option in selected tumors in multiple organs such as the liver, lung, pancreas, kidney, and prostate and benign indications such as abnormal uterine bleeding (AUB), benign prostatic hypertrophy (BPH), cardiac arrhythmias. Focal ablation requires precisely dosed and accurate targeting of the tissue to be ablated while preserving surrounding healthy tissues and vital structures such as blood vessels, nerves, and neighboring organs. The unselective destruction inflicted by thermal ablation modalities can result in damage to vital structures in the vicinity of the targeted diseased tissue. Furthermore, the efficacy of thermal ablation intensity can be impaired due to “thermal sink”. Close proximity of large vessels, bile ducts, or the renal collecting system can cause thermal fluctuations, leading to inconsistent ablation results.

The theory of cell membrane poration, RE and IRE are not dependent on thermal energy for tissue damage and is therefore not influenced by “thermal sink”, promising consistent results in the vicinity of large vessels or the renal collecting system. IRE lesions typically show a sharp demarcation between ablated and nonablated tissues, whereas thermal ablation techniques show a transitional zone of partially damaged tissue wherein insufficient temperatures were reached for definitive ablation. This indicates that IRE ablation boundaries may potentially be planned more precisely compared to conventional thermal techniques.

A commercially available IRE console for clinical use in tissue ablation consists of a low-energy direct current generator interfaced with a computer system and has the capability of connecting up to six monopolar needle electrodes, 16 G in diameter and covered in a retractable insulation sheath, allowing for the adjustment of the active tip length. Early animal research showed bipolar IRE to result in lower ablation volumes, combined with a higher risk of collateral damage. Currently practiced IRE settings for tumor ablation are voltage 1,500 V/cm, 70-90 pulses of 70-90 μs, electrode spacing 1.5-2 cm, and active electrode tip length 1-1.5 cm. In contrast, monopolar IRE uses high-intensity electrical pulses causing severe muscle contractions hence require paralytic agents and general anesthesia. In addition, administered IRE pulses could potentially cause cardiac arrhythmia, depending on the distance of the ablation spot to the heart.

Additionally, so far, it has been difficult for IRE to completely ablate large tumors, for example tumors that are >3 cm in diameter. To overcome this problem, many preclinical and clinical studies have been performed to improve the efficacy of IRE in the treatment of large size of tumors through a chemical perspective. Due to the distribution of electric field, IRE region, RE region, and intact region can be found in the treatment of IRE. Chemical potentiation converts RE zone into IRE zone however, cytotoxic chemicals like chemotherapeutic agents such as cisplatin, carboplatin and bleomycin cannot be used for benign indications. Also it's difficult to introduce chemical agents into live tissues in adequate therapeutic concentration to create a reliable therapeutic concentration. The efficacy of chemosensitization diminishes in tumor>3 cm where its most needed. The side effects of ECT with large dose of cytotoxic drugs while treating large tumors are of severity in clinical practice, such as pulmonary fibrosis, particularly when this treatment is administered to patients who have previously received radiation therapy. It is also clinically difficult to inject intratumor due to the risk of dissemination of tumor cells or seeding of the needle track with tumor. Calcium electroporation is another method of chemical enhancement however, its mechanism is not fully understood and efficacy in the treatment of large and heterogeneous tumors is unknown. Chemical modification of cell membranes using DSMO and SDS has been attempted however, clinical safety of such agents is unknown and there is lack of human clinical data. Additionally, there are very few surfactants available. Finally, the heterogeneity of tumor tissues needs to be considered and remains a challenge while introducing chemical drugs or ions into benign and tumor tissues during IRE.

Hence there is a need for improved technologies to enhance the therapeutic effects of nonthermal electroporation while maintaining its safety margin within an RE region. There is also a need to optimally ablate an entire area of a target tissue.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a system for ablation, comprising: a catheter with a proximal end, a distal end, and a lumen, comprising: a thermal chamber in fluid communication with the lumen; at least one first positioning element positioned proximate the distal end of the catheter; at least one delivery port positioned proximate the distal end of the catheter and configured to deliver thermal energy generated within the thermal chamber; and at least one electrode positioned proximate the distal end of the catheter and configured to generate an electrical field to cause electroporation when activated; and a controller comprising a microprocessor for controlling the delivery of the thermal energy and the generating of the electrical field.

Optionally, the thermal chamber is positioned within the lumen.

Optionally, a size of a thermal field created by thermal energy is a similar size, shape, or volume as the electrical field.

Optionally, a size of a thermal field created by thermal energy is a different size than the electrical field.

Optionally, the at least one first positioning element comprises any one of a wire mesh, disc, hood, cap, or inflatable balloon.

Optionally, the at least one first positioning element comprises any one of a circular, oval, rectangular, conical, spherical, oblong, or square shape.

Optionally, the system further comprises at least one needle at the distal end of the catheter wherein the at least one needle includes the at least one delivery port.

Optionally, the at least one electrode configured to generate an electrical field is positioned on the at least one first positioning element. Optionally, the at least one electrode configured to generate an electrical field is printed on a surface of the at least one first positioning element.

Optionally, the system further comprises at least one second positioning element proximate a distal end of catheter. Optionally, the at least one second positioning element comprises any one of a wire mesh, disc, hood, cap, or inflatable balloon. Optionally, the at least one first positioning element comprises any one of a circular, oval, rectangular, conical, spherical, oblong, or square shape. Optionally, the at least one delivery port configured to deliver thermal energy is positioned on the catheter between the at least one first positioning element and the at least one second positioning element.

The present specification also discloses a method for ablation of a target tissue area, comprising: inserting a catheter comprising at its distal end at least one positioning element, at least one port proximate the at least one positioning element configured to deliver thermal energy, and at least one electrode proximate the at least one positioning element configured to generate an electric field to cause electroporation; deploying the distal end proximate the target tissue area; applying a combination of thermal energy from the at least one port and an electrical field from the at least one electrode.

Optionally, thermal energy is applied before applying the electrical field.

Optionally, thermal energy is applied at the same time that the electrical field is applied.

Optionally, thermal energy is applied after applying the electrical field.

Optionally, a temperature of the target tissue area is raised to greater than 40° C. for more than 1 second.

Optionally, a temperature of the target tissue area is raised to greater than 40° C. and less than 100° C. for more than 1 second.

Optionally, a temperature of the target tissue area is lowered to less than 25° C. and greater than −200° C. for more than 1 second.

Optionally, a temperature of the target issue area is raised to greater than 100° C. for less than 1 second.

Optionally, a temperature of the target issue area is raised to greater than 110° C. for less than 1 second.

Optionally, thermal energy is applied to the tissue for a duration between 1 second and 30 minutes.

Optionally, electrical energy is applied to the tissue for a duration less than 1 second.

Optionally, a pressure at the target tissue area is not increased above 5 atm.

Optionally, a temperature of the target tissue area is altered within five minutes of applying the electrical field.

Optionally, a thermal energy is applied to raise a temperature of the target tissue area is to greater than 40° C. and less than 100° C. for more than 1 second and an electrical energy field is applied to raise a temperature of the target tissue area to greater than 100° C. for less than 1 second.

Optionally, a thermal energy is applied to lower a temperature of the target tissue area is to less than 25° C. for more than 1 second and an electrical energy field is applied to raise a temperature of the target tissue area to greater than 100° C. for less than 1 second.

Optionally, a thermal energy is applied to raise a temperature of the target tissue area to greater than 40° C. and less than 100° C. for more than 1 second and the electric field is applied for electroporation for less than 1 second to cause an irreversible tissue change.

Optionally, a thermal energy is applied to lower a temperature of the target tissue area to less than 25° C. for more than 1 second and the electric field is applied for electroporation for less than 1 second to cause an irreversible tissue change.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a comparative graphical representation of electrical pulses conventionally used in electroporation and high-frequency electroporation methods;

FIG. 2 illustrates an ideal schematic of electric fields distribution around electroporation electrodes, in accordance with some embodiments of the present specification;

FIG. 3A illustrates positioning of two needle-based catheter devices used for combining delivery of thermal energy and electroporation, inside a human organ, in accordance with some embodiments of the present specification;

FIG. 3C illustrates a system for use in controlling the delivery of the thermal agent and electrical pulses, and also enabling the physician to control the treatment, in accordance with some embodiments of the present specification;

FIG. 3B is a schematic representation of a needle-based catheter of FIG. 3A, in accordance with some embodiments of the present specification;

FIG. 4A is a schematic representation of a balloon-based catheter device used for combining delivery of thermal energy and electroporation, in accordance with some embodiments of the present specification;

FIG. 4B is an illustration of a distal end of the balloon-based catheter device of FIG. 4A;

FIG. 5A illustrates a balloon positioning element attached to a distal end of a catheter with printed surface electrode for sensing of physiological signals as well as delivery of electroporation electrical field;

FIG. 5B illustrates a cross sectional view of a mid-shaft portion of the catheter of FIG. 5A with embedded electrode wires in the catheter wall for sensing of physiological signals as well as delivery of electroporation electrical field;

FIG. 5C illustrates a cross sectional view of a distal tip portion of catheter of FIG. 5A with a central lumen for ablation of thermal energy as well as passage of sensing catheter and contrast agent;

FIG. 6A is a schematic representation of another configuration of a catheter with a pyramidical positioning element at its distal end, in accordance with some embodiments of the present specification;

FIG. 6B illustrates an exemplary area of an IRE field created by electrodes of FIG. 6A, and an overlapping thermal field created by the heater vapor delivered by thermal port;

FIG. 7 is a schematic representation of yet another configuration of a catheter with a conical positioning element at its distal end, and a needle thermal delivery port in the center, in accordance with some embodiments of the present specification;

FIG. 8 illustrates still another embodiment of a catheter with at least two positioning elements at its distal end with electroporation electrodes (not shown) located on the at least two positioning elements, in accordance with some embodiments of the present specification;

FIG. 9 is a schematic representation of various sequences of applying a thermal energy and an electrical field for electroporation in accordance with various embodiments of the present specification;

FIG. 10 is a flow chart illustrating an exemplary process of ablation in accordance with some embodiments of the present specification;

FIG. 11 is a flow chart illustrating another exemplary process of ablation in accordance with an embodiment of the present specification;

FIG. 12 is a flow chart illustrating yet another exemplary process of ablation in accordance with an embodiment of the present specification; and

FIG. 13 is a flow chart illustrating still another exemplary process of ablation in accordance with an embodiment of the present specification.

### DETAILED DESCRIPTION

Controlled application of thermal energy proximate an application of non-thermal electroporation enhances the IRE/RE region ratio. Thermally induced structural or functional changes occur to the membrane lipids which deforms the lipid tails and increases permeability of the bilayer to water, ions, and small molecules. Thermally induced modulation of membrane proteins occurs, especially for a voltage-gated channel that allows ion transportation across the membrane. These changes are incremental and additive to those induced by the electrical field and either prolong the duration of reversible change or convert a reversible change into an irreversible change resulting in an irreversible damage to cellular structures which is greater than that created by the electrical or thermal field alone.

The present specification is directed toward application of controlled thermal energy in conjunction with electrical energy such as electroporation to increase the irreversible electroporation (IRE) region by converting the reversible electroporation (RE) region that may surround the IRE region, to an IRE region. The application of thermal energy between the ranges of 45° C. and 100° C. or <25° C. before, after, or concurrent with electroporation energy does not significantly interfere with electrical electroporation energy field and energy delivery. Embodiments provide methods and systems for controlled application of thermal energy along with application of electrical or electroporation energy to enhance the ratio of IRE to RE region and hence the coagulation or ablation effect. In some embodiments, the thermal energy is delivered prior to delivering the electrical field or the electroporation pulse. In some embodiments, the thermal energy is delivered concomitant to delivering the electrical field or the electroporation pulse. In embodiments, thermal energy is applied within 30 minutes of the electrical field or the electroporation energy for optimal lesion creation. Further, application of thermal energy in accordance with the present specification, decreases one of the capacitor size, peak voltage, or pulse width requirement of an electrical field or an electroporation treatment. In some embodiments, the combination improves the efficacy of thermal ablation without worsening the safety profile of the thermal lesion. In other embodiments, the combination improves the efficacy of thermal ablation while improving the safety profile of the thermal lesion.

In some embodiments, the combination decreases the duration of treatment without worsening the efficacy or safety profile of the thermal lesion. In other embodiments, the combination does not significantly increase the duration of thermal ablation while improving the efficacy or safety profile of the thermal lesion.

A combination of thermal and non-thermal ablation is applied to ablate a minimum portion of a target surface area or volume optimally. In some embodiments, the minimal area of a target tissue that is required to be optimally ablated is approximately 50%. In embodiments, methods and systems of the present specification achieve approximately 25% optimal ablation using one method and the remaining 25% using another. In one embodiment, a therapeutic dose of thermal ablation is delivered to therapeutically ablate at least 25% of the surface area or the volume. Then Pulse Field Ablation (PFA) or an electroporation method is used to therapeutically ablate over 50% of the surface area without over-ablating any of the first at least 25% of the thermally ablated area, wherein over-ablating is defined as ablating adjacent normal tissue resulting in complications without improved efficacy. In another embodiment, a therapeutic dose of PFA or electroporation method is used to therapeutically ablate at least 25% of the target surface area. This is followed by delivering thermal ablation to therapeutically ablate over 50% of the target surface area without over-ablating any of the first at least 25% of the non-thermally ablated area. In another embodiment, the combination of electrical field and the thermal field results in a cumulative surface area or volume of ablation which is more than that obtained by either of the individual modalities resulting in an additive or synergistic ablation effect.

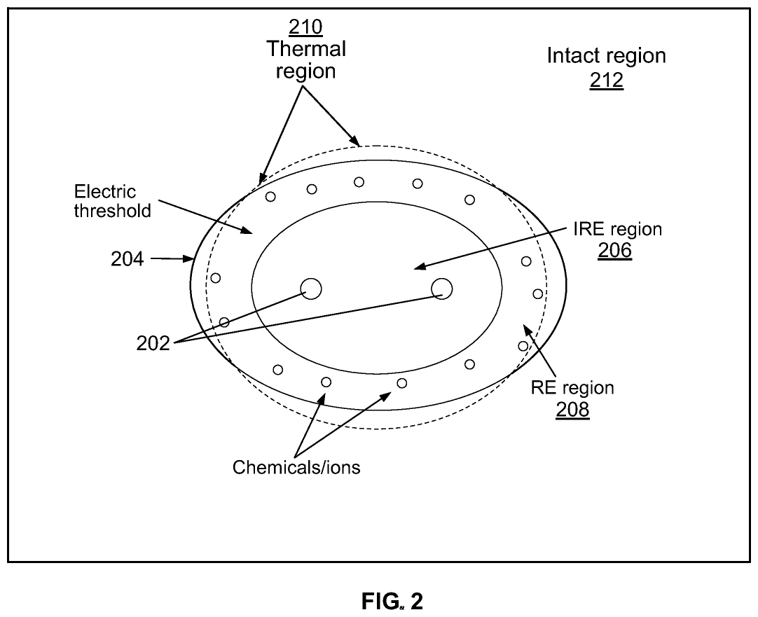

In some embodiments, the thermal energy is delivered using heated water vapor using delivery tip configuration in accordance with the present specification, so that the thermal field roughly overlaps the electrical field created by electroporation pulses and in most cases exceed the IRE field and covers a portion or whole of the RE field. FIG. 2 illustrates an ideal schematic of electric fields distribution around bipolar electrodes 202, in accordance with some embodiments of the present specification. A tissue area 204 is targeted by electrodes 202, which creates an IRE region 206 (IRE electrical field created by electroporation pulses), surrounded by an RE region 208 (RE electrical field created by electroporation pulses). Introduction of thermal energy, such as in the form of heated water vapor or hot water or a cryogen, creates a thermal region 210. Similar to the electroporation field comprising IRE region 206 and RE region 208, the thermal region 210 has an irreversible thermal injury zone and a reversible thermal injury zone. Thermal region 210 overlaps IRE region 206 and RE region 208, such that thermal region 210 is greater than IRE region 206 and either partially or fully overlaps RE region 208. Area 212 surrounding regions 206, 208, and 210 remains intact and unaffected by the electroporation energy and the thermal energy. The intersection of reversible electroporation and reversible thermal injury creates a new therapeutic zone of irreversible injury or ablation.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications, and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

The term “controller” refers to an integrated hardware and software system defined by a plurality of processing elements, such as integrated circuits, application specific integrated circuits, and/or field programmable gate arrays, in data communication with memory elements, such as random-access memory or read only memory where one or more processing elements are configured to execute programmatic instructions stored in one or more memory elements.

The term “vapor generation system” refers to any or all of the heater or induction-based approaches to generating steam from water described in this application.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

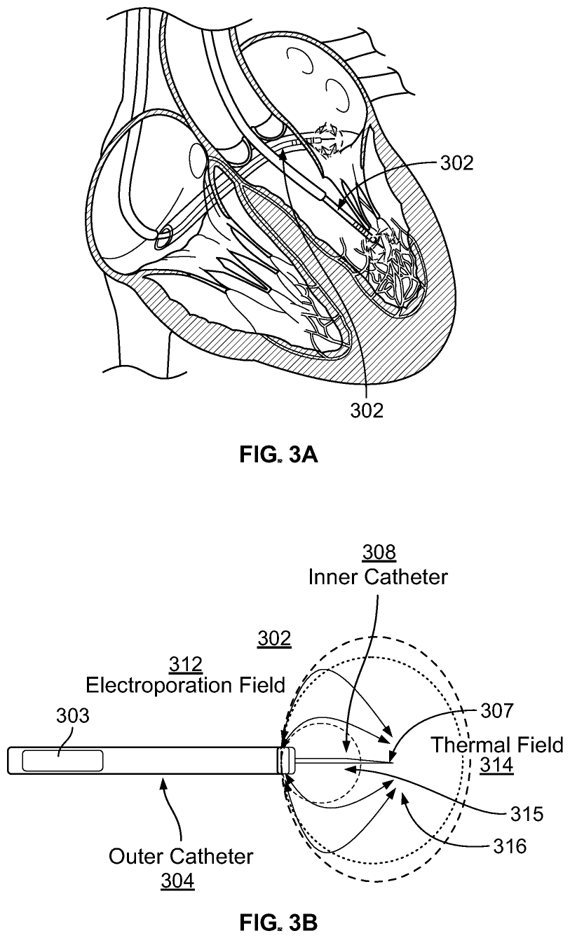

FIG. 3A illustrates positioning of two needle-based catheter devices 302 used for combining delivery of thermal energy and electroporation, inside a human organ, in accordance with some embodiments of the present specification. FIG. 3B is a schematic representation of a needle-based catheter 302 of FIG. 3A, in accordance with some embodiments of the present specification. Device 302 includes an outer catheter 304 comprising an elongated tube with a lumen. In some embodiments, catheter 304 may include two lumens—one for delivery of heated fluid or vapor, and another for a guidewire. Catheter 304 is a coaxial needle catheter. An inner catheter 308 is comprised coaxially within outer catheter 304, and comprises an inner lumen that delivers the ablative fluid, a contrast and the guidewire. An inner needle 307 extends out from the inner catheter 308, and has a metallic tip which acts as a pole/electrode for bipolar IRE delivery and for sensing of electrical activity or measuring another physiological tissue parameter. Outer catheter 304 has at least one distal attachment or positioning element. Positioning element may be any one of a wire mesh, disc, hood, cap, or inflatable balloon. An outer sheath of catheter 304 has electrodes for sensing and delivery of IRE. In some embodiments, a thermal or heating chamber is configured to heat a fluid provided to outer catheter 304 to change the fluid to heated water or water vapor for delivering thermal energy. In embodiments, the thermal or heating chamber is in fluid communication with the lumen of the outer catheter 304. In some embodiments, the thermal or heating chamber is an internal thermal or heating chamber 303 and is disposed within the lumen of outer catheter 304. In some embodiments, outer catheter 304 is made of or covered with an insulated material to prevent the escape of thermal energy from the catheter body. Fluid, such as saline, is stored in a reservoir and delivered through the lumen using a saline pump connected to catheter 304.

Delivery of the thermal agent is controlled by a controller and treatment is controlled by a treating physician via the controller. The controller includes at least one processor in data communication with the saline pump and a catheter connection port in fluid communication with the saline pump. In some embodiments, at least one optional sensor monitors changes in a thermal region to guide flow of thermal agent. Catheter 304 has embedded wire with plurality of exposed electrodes at the end for both IRE delivery and sensing of any physiological parameter such as electrical activity, tissue impedance, tissue temperature, or any other physiological parameter. In some embodiments, the electrodes are configured to sense and deliver electroporation sequentially, and sense simultaneously during delivery of an ablative agent. In some other embodiments, an EP catheter is delivered through a second lumen within outer catheter 304, which can sense simultaneous to the delivery of electroporation. In yet other embodiments, the embedded catheter wires comprise two separate circuits—one for sensing and one for delivery of electroporation. In some embodiments, the optional sensor comprises at least one of a temperature sensor or pressure sensor. In some embodiments, catheter 304 includes a filter with micro-pores which provides back pressure to the delivered water vapor, thereby pressurizing the steam. The predetermined size of micro-pores in the filter determine the backpressure and hence the temperature of the steam being generated.

In one embodiment, a user interface included with the controller allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

Inner catheter 308 is comprised coaxially within outer catheter 304. Inner catheter 308 includes a distal positioning element (not shown) and a thermal energy delivery port 307. In embodiments, inner catheter 308 has a needle-shaped tip and includes a lumen for delivery of thermal energy through thermal energy delivery port 307. In some embodiments, the inner needle has a metallic tip which act as a pole/electrode for bipolar electroporation delivery and for sensing of electrical activity or measuring another physiological tissue parameter. One or more electrodes (not shown) are located on each positioning element at distal end of outer catheter 304. The electrodes are electrically insulated from each other. Electrical pulses are applied for electroporation, between the two electrodes while thermal energy is applied from thermal energy delivery ports to create a thermal field 314, so that thermal field 314 roughly overlaps the electrical field created by electroporation pulses. Electrical field includes an electroporation field 312 which is composed of an IRE field 315 and an RE field 316, and in most cases thermal field 314 exceeds IRE field 315 and covers a portion or whole of RE field 316. As shown in FIG. 3B, IRE field 312 (shown by the arrows in FIG. 3B) created by the electrodes of the positioning elements approximates a thermal energy field 314 created by the delivery of thermal energy.

The application of thermal energy decreases at least a voltage or an amplitude or a pulse width or a duration of electroporation electrical pulse sourced by the electrodes, to achieve irreversible damage to a volume of tissue within electroporation field 312. In one embodiment the IRE electrical pulse is delivered at a peak voltage 1 kV-6 kV and a pulse width of 0.5 μs-10 μs. Additionally, capacitor size in a range of 500 μF to 5 mF are used with the electroporation electrodes. In one embodiment the electroporation electrical pulse is a monophasic pulse. In another embodiment the electroporation electrical pulse is a biphasic pulse. In another embodiment the electroporation pulse is a triphasic pulse.

FIG. 3C illustrates a generator 300c for use in controlling the delivery of the thermal agent and electrical pulses, and also enabling the physician to control the treatment, in accordance with some embodiments of the present specification. Generator 300c is connected to catheter devices 302 used for combining delivery of thermal energy and electroporation. In some embodiments, the connection between system 300c and catheter devices is interfaced through a handle (not shown) that includes actuators/buttons/knobs to control various parameters of thermal agent and electroporation, and start and stop of treatment. A cable sub-assembly can be provided which includes an electrical cable in the handle to connect catheter device 302 to generator 300c.

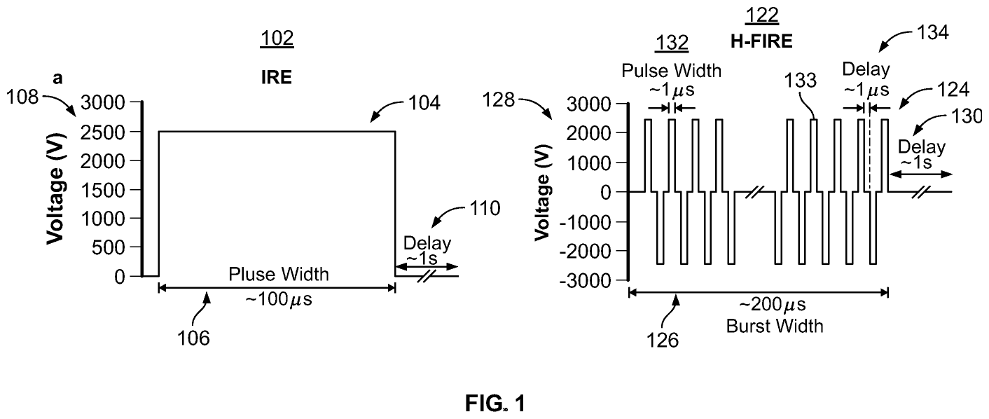

Generator 300c controls the delivery of the ablative agent to the vapor ablation system and electrical pulses to the electroporation system. Referring again to FIG. 1, some embodiments of generation of electrical pulses are described which are generated by pulse generator within generator 300c. The generator 300c therefore provides a control interface to a physician for controlling the treatment. An input port 302c on generator 300c provides a port to connect generator 300c to the catheter and provide electrical signal to the catheter. A fluid port 304c provides a port for connecting a supply to fluid such as saline or water through a tubing to the catheter. In embodiments, a graphical user interface (GUI) 306c shows the settings for operating the ablation system and the electroporation system, which may be in use and/or modified by the physician during use. In some embodiments, the GUI is a touchscreen allowing for control of generator 300c by a user.

In embodiments, the GUI provides a user control to define an extent or a percentage of physical overlap between the different treatment combinations, which can be defined in terms of time of thermal treatment and electroporation treatment. For example, referring to FIGS. 2, 3B, and 3C, in embodiments, the generator 300c includes controls, such as via the GUI 306c, to define and control the extent or percentage of physical overlap between the thermal region 210, RE region 208, and IRE region 206 as seen in FIG. 2, or the IRE field 315 and RE field 316 of the electroporation field 312, and the thermal field 314 as seen in FIG. 3B. Therefore, in various embodiments, the generator 300c provides control to the operator for defining the extent or percentage of physical overlap between four combinations of therapy: reversible thermal therapy with RE, irreversible thermal therapy with IRE, irreversible thermal therapy with RE, and reversible thermal therapy with IRE. In various embodiments, the electrical fields (IRE and RE fields) and thermal fields (reversible thermal field and irreversible thermal field) are of any size, depending on energy and time as delivered by the generator/controller, and generally have a spherical shape. The shape of the electrical fields will not be exactly the same as the thermal fields because heat and electricity travel differently through the heterogenous tissue layers. Both the electrical fields and thermal fields will have an irreversible zone at the center of each field and a reversible zone in the periphery. The size of the irreversible zone will determine the desired therapeutic effect. Where the reversible zones intersect, there will a zone of irreversible effect which will have an enhanced therapeutic effect. By combining the two reversible zones, the systems and methods of the present specification improve the therapeutic effect without increasing the complications. In some embodiments, there is ideally at least a 25% overlap between the reversible zone of the electrical field and the reversible zone of the thermal field. In other embodiments, there is at least a 10% overlap between the reversible zone of the electrical field and the reversible zone of the thermal field. In some embodiments, there is ideally at least a 50% overlap between the total electrical field and the total thermal field. In some embodiments, there is at least a 25% overlap between the total electrical field and the total thermal field.

Additionally, there may be reversible islands in the irreversible zone of both electrical fields and the thermal fields which will not be in the same area. In thermal therapy or electroporation alone, these islands of reversible damage will results in therapeutic failure. By combining the two zones, the systems and methods of the present specification act to minimize or eliminate these reversible islands.

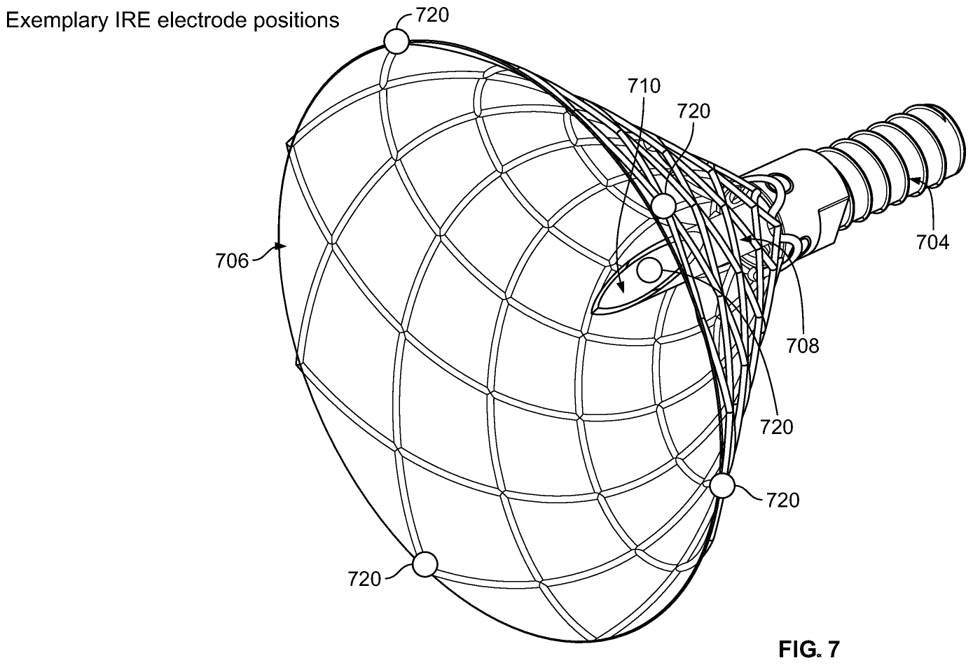

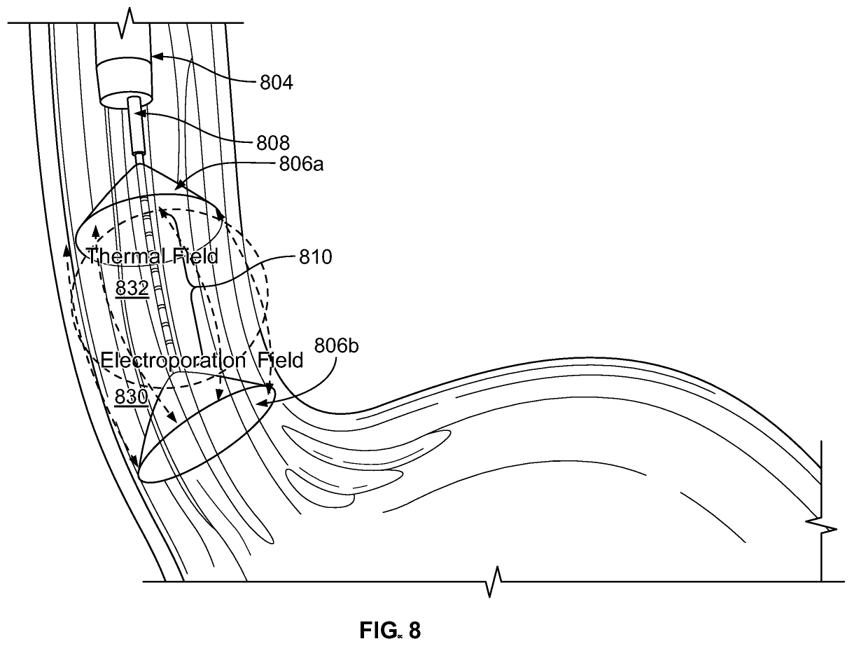

Further, FIG. 9 provides details of different types of combinations and a schematic representation of various sequences of applying a thermal energy and an electrical field for electroporation in accordance with various embodiments of the present specification. Additionally, FIGS. 10-13 illustrate exemplary methods using different types of thermal and electrical ablation methods, which are controlled using generator 300c. In embodiments, as the time is changed, the GUI is configured to display a corresponding visual output of the extent of physical coverage of treatment and overlap of thermal and electroporation treatments.

In various embodiments, generator 300c of the present specification comprises a computing device having one or more processors or central processing units, one or more computer-readable storage media such as RAM, hard disk, or any other optical or magnetic media, a controller such as an input/output controller, at least one communication interface and a system memory. The system memory includes at least one random access memory (RAM) and at least one read-only memory (ROM). In embodiments, the memory includes a database for storing raw data, images, and data related to these images. The plurality of functional and operational elements is in communication with the central processing unit (CPU) to enable operation of the computing device. In various embodiments, the computing device may be a conventional standalone computer or alternatively, the functions of the computing device may be distributed across a network of multiple computer systems and architectures and/or a cloud computing system. In some embodiments, execution of a plurality of sequences of programmatic instructions or code, which are stored in one or more non-volatile memories, enable or cause the CPU of the computing device to perform various functions and processes as described in the present specification. In alternate embodiments, hard-wired circuitry may be used in place of, or in combination with, software instructions for implementation of the processes of systems and methods described in this application. Thus, the systems and methods described are not limited to any specific combination of hardware and software.

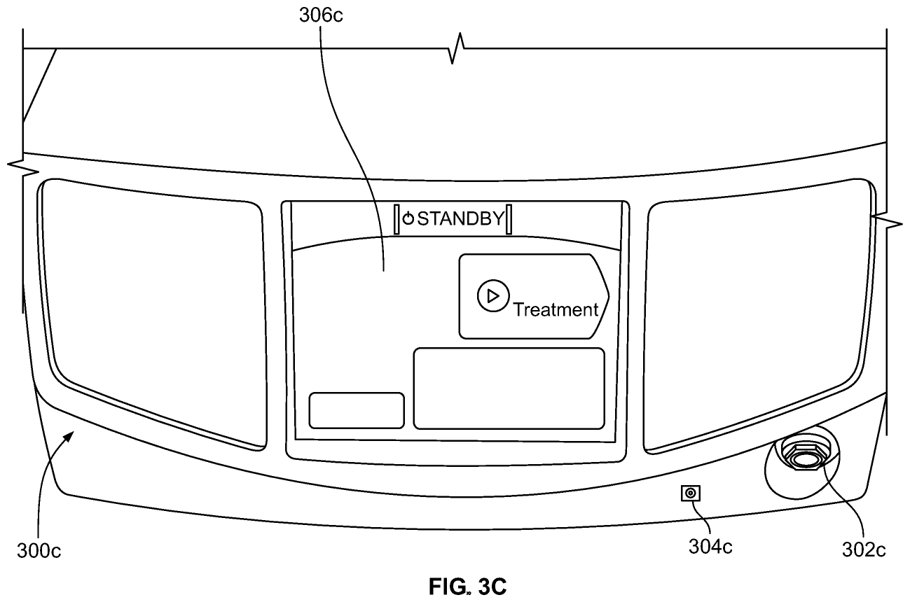

FIG. 4A is a schematic representation of a system comprising a generator 400c and a balloon-based catheter device 402 used for combining delivery of thermal energy and electroporation, in accordance with some embodiments of the present specification. FIG. 4B is an illustration of a distal end of the balloon-based catheter device 402 of FIG. 4A. In embodiments, generator 400c (equivalent to generator 300c of FIG. 3C) is in communication with catheter device 402 and is configured to control the treatment delivered using thermal ablation and electroporation. An electrical cable 422 connects a port (port 302c) to provide electrical signal to catheter device 402, preferably in some embodiments to one or more electrodes within an outer catheter 404. Another cable/tube 424 connects a fluid port (port 304c) for connecting a supply of fluid such as water or saline to catheter device 402, preferably in some embodiments to infusion ports in an inner catheter 408. In some embodiments, a thermal or heating chamber is configured to heat a fluid provided to inner catheter 408 to change the fluid to heated water or water vapor for delivering thermal energy. In embodiments, the thermal or heating chamber is in fluid communication with the lumen of the inner catheter 408. In some embodiments, the thermal or heating chamber is an internal thermal or heating chamber 407 and is disposed within a lumen of inner catheter 408. In embodiments, two positioning elements (balloons) 406 and 410 are attached to two different lumens at the distal end of catheter device 402. An outer balloon 406 comprises one or more electrodes 420 positioned on its surface. FIG. 4B illustrates a printed electrodes 420 configuration. In some embodiments, electrodes 420 are a plurality of printed electrodes for electroporation and are electrically insulated from each other. Electroporation pulses are applied between one or more electrodes 420 while the thermal energy is applied from infusion ports in inner catheter 408 surrounded by an inner balloon 410. Inner balloon 410 is encompassed by outer balloon 406, so thermal energy from the heated water vapor passes from inner balloon 410 through the outer balloon 406, wherein an electroporation field 412 approximates a thermal energy field 414 as the areas of the thermal contact and electrical contact approximate each other. Embodiments of FIGS. 4A and 4B may be used with electroporation or pulsed field ablation (PFA) systems in cardiac ablation. In cardiac applications, electroporation is applied in a single shot using any combination of parameters. In one embodiment, the combination and intersection of the electroporation and thermal field creates the majority of the thermal lesion. Various configuration of double-balloon structures described in the art to create a thermal lesion can be used to deliver the thermal energy. The system can be used for ablation of any vascular structure including an artery or vein, including a pulmonary artery or a pulmonary vein or a renal artery or a renal vein.

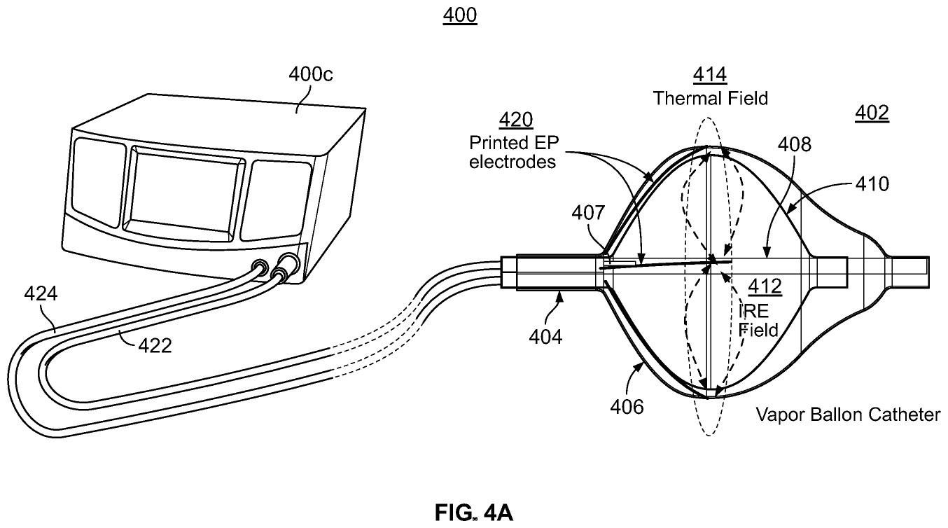

FIG. 5A illustrates an inflatable balloon 506 positioning element attached to a distal end of a catheter 504, such as positioning element 406 attached to outer catheter 404 of FIG. 4A. Surface of balloon 506 includes electroporation electrodes 520 of catheter 504. FIG. 5B illustrates a cross sectional view of a mid-shaft portion of the catheter 504 with embedded electrode wires in the catheter wall for sensing of physiological signals as well as delivery of electroporation electrical field. The catheter 504 includes a compartmentalized outer wall 2068 which includes the air/water lumen 2062 and conductive wires 2069 connected to electrodes 520. The catheter 504 also includes the vapor lumen 2063 and, in one embodiment, a guidewire lumen 2070. In certain embodiments, the vapor lumen 2063 and the guidewire lumen 2070 are the same. FIG. 5C illustrates a cross sectional view of a distal tip portion of catheter 504 with central lumen for ablation of thermal energy as well as passage of sensing catheter or a guide wire and contrast agent. The catheter 504 includes a plurality of electrodes 520 built into its outer wall or, in an embodiment, into the wall of the balloon 506, the vapor lumen 2063, and a guidewire lumen 2070.

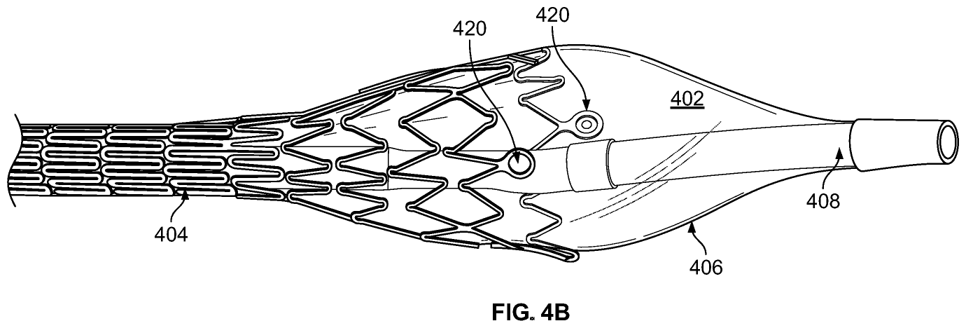

FIG. 6A is a schematic representation of another configuration of a catheter 604 with a pyramidical positioning element 606 at its distal end, in accordance with some embodiments of the present specification. Positioning element 606 has either a square or a rectangular base that is open, while the tip at a proximal side of the pyramid converges towards and is attached to distal end of outer catheter 604. In other embodiments, the positioning element has a circular, oval, conical, spherical, oblong, or square shape In embodiments, dimension of positioning element 606 have a known length ‘l’ and side ‘d’, which are used to calculate an amount of thermal energy needed to achieve a target tissue temperature. The sloping surfaces of positioning element 606 is covered by an optional insulated membrane that prevents escape of thermal energy or vapor away from a target site. Outer catheter 604 includes a lumen that comprises a coaxial inner catheter 608 configured to deliver heated water vapor from a thermal port 610 at its distal end. A plurality of electrodes 620 are positioned on a surface of positioning element 606. In some embodiments, an electrode is positioned on each corner of the base of pyramidical positioning element 606. Optionally, an electrode 622 is positioned proximate thermal port 610 in a central region of positioning element 606. During operation, electroporation pulses are applied between the electrodes 620 and 622, while thermal energy is applied from thermal port 610. A resulting electroporation field 630 approximates the thermal energy field 632 created by the delivery of heated water vapor from thermal port 610 or any other ablative agent or energy source such as a cryogen or plasma energy. FIG. 6B illustrates an exemplary area of an IRE field 630 created by electrodes 620 and 622, and an overlapping thermal field 632 created by the heated vapor or another ablative agent delivered via thermal port 610.

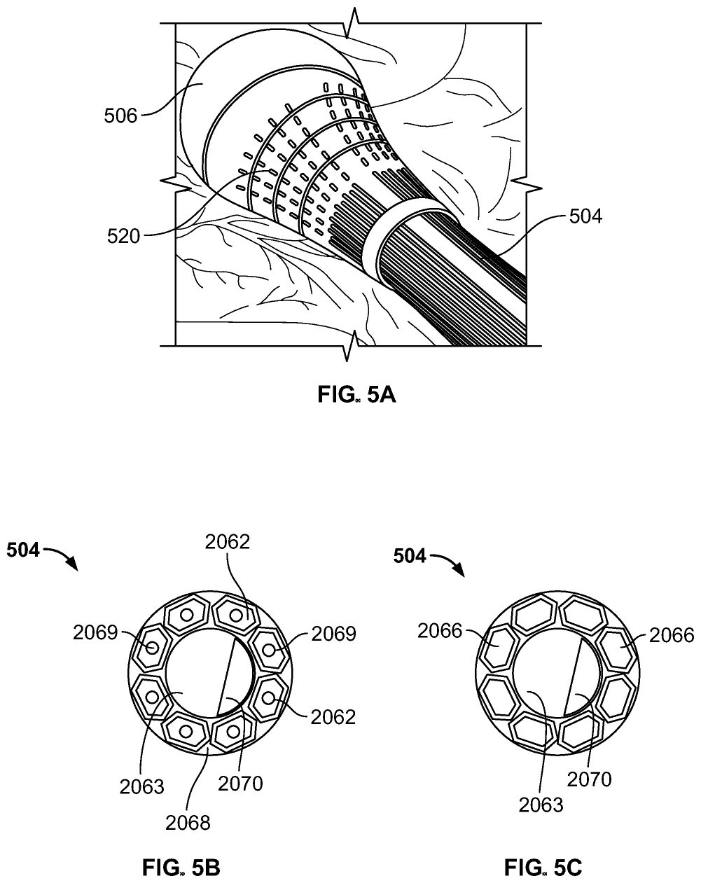

FIG. 7 is a schematic representation of yet another configuration of a catheter 704 with a conical positioning element 706 at its distal end, in accordance with some embodiments of the present specification. Positioning element 706 has either a circular base that is open, while the tip at a proximal side of the pyramid converges towards and is attached to distal end of outer catheter 704. In some embodiments, the shape of the positioning element 706 is oval. In embodiments, dimension of positioning element 706 have a known length ‘l’ and base diameter ‘d’, which are used to calculate an amount of thermal energy needed to achieve a target temperature. The sloping surfaces of positioning element 706 is covered by an optional insulated membrane that prevents escape of thermal energy or vapor away from a target site. Outer catheter 704 includes a lumen that comprises a coaxial inner catheter 708 configured to deliver heated water vapor from a thermal port 710 at its distal end. A plurality of electrodes 720 are positioned on a surface of positioning element 706. In some embodiments, at least four equally spaced electrodes 720 are positioned proximate the circular distal edge of positioning element 706. Optionally, an electrode 722 is positioned proximate thermal port 710 in a central region of positioning element 706. During operation, IRE pulses are applied between the electrodes 720 and 722, while thermal energy is applied from thermal port 710. In one embodiment thermal port 710 is shaped like a needle to puncture the tissue and deliver the ablative agent into the tissue.

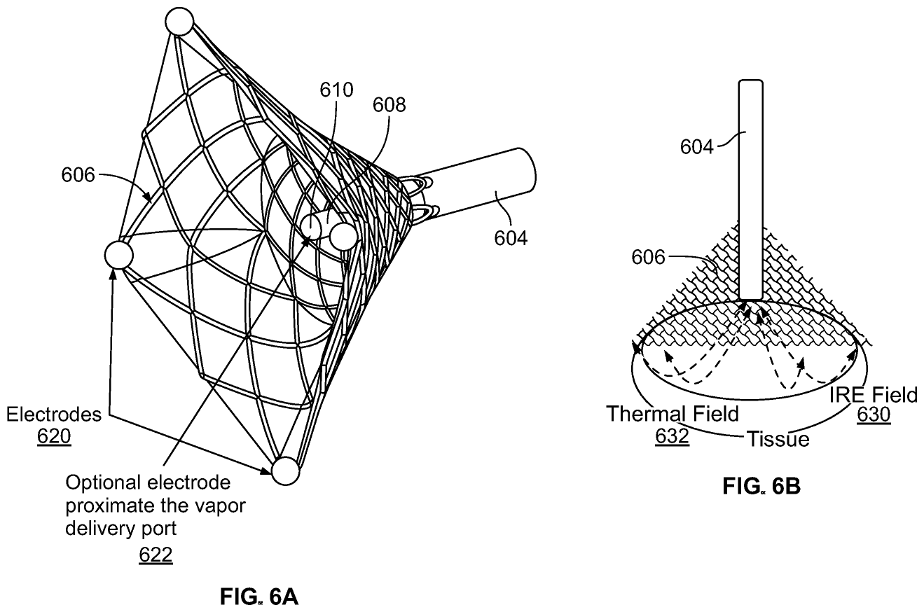

FIG. 8 illustrates still another embodiment of a catheter 804 with at least two positioning elements 806a and 806b at its distal end with electroporation electrodes (not shown) located on the at least two positioning elements, in accordance with some embodiments of the present specification. In some embodiments, the number of positioning elements is different. In all embodiments, the plurality of positioning elements are proximate a plurality of thermal energy delivery ports, and each positioning element has one or more electroporation electrodes located with them. Referring to FIG. 8, electroporation pulses are applied between the two positioning elements 806a and 806b by operating the electrodes, which create an electroporation field 830. Thermal energy is supplied through inner catheter 808 and delivered as heated vapor through multiple ports 810, and which creates a thermal field 832. Ports 810 are connected to distal end of inner catheter 808 and are located between the two positioning elements 806a and 806b. Electroporation field 830 and thermal field 832 are generated between the two positioning elements 806a and 806b. Further, electroporation field 830 approximates thermal field 832. Embodiments of FIG. 8 may be preferred for deployment in an air filled tubular organ such as the tracheal tube of a patient.

In the above embodiments the temperature of the targeted tissue is altered by delivery of thermal energy within 30 minutes of applying electroporation electrical field. In some of the embodiments the temperature of the targeted tissue is altered within five minutes of applying the electroporation electrical field. In some of the embodiments the temperature of the targeted tissue is altered within one minute of applying the electroporation electrical field. In some embodiments, the thermal energy is delivered prior to delivering the electroporation pulse. In some other embodiments, the thermal energy is delivered after delivering the electroporation pulse. In yet other embodiments, the thermal energy is delivered concurrently with the application of the electroporation field. In various embodiment the thermal energy serve the function of priming the targeted tissue to improve one of the safety or efficacy of the electroporation field or vice-versa.

FIG. 9 is a schematic representation of various sequences of applying a thermal energy and an electrical field for electroporation in accordance with various embodiments of the present specification. A first graph 902 represents application of thermal energy followed by application of IRE field. A second graph 904 represents concurrent application of the thermal energy and the electrical field for electroporation. A third graph 906 represents application of an electrical field for IRE followed by the thermal energy. In all the cases, the duration of applying the thermal energy is greater than that of applying the electric field for electroporation. In an embodiment, the thermal energy is applied for more than 1 second and electrical field for electroporation is applied for less than 1 second. In various embodiments, the temperature of the target tissue is raised to one of the following levels during the therapy session: greater than 40° C. for more than 1 second, greater than 50° C. for more than 1 second, greater than 60° C. for more than 1 second, greater than 70° C. for more than 1 second, greater than 80° C. for more than 1 second, greater than 90° C. for more than 1 second, greater than 100° C. for 1 second or more than 1 second. The target temperature setting may be provided by a user or a physician through the controller connected to the catheter. In some embodiments, sensors deployed within the system are used to monitor target temperatures, which are in turn used to control the duration and power of delivering the thermal energy and the electrical field. Alternatively, in some embodiments, the target temperatures are measured during the dosimetry studies to identify the ideal duration and power of delivering the thermal energy and the electrical field.

In some embodiments, combining delivery of thermal energy with electroporation increases the volume of tissue that is irreversibly damaged as compared to the volume of tissue irreversibly damaged by IRE alone. In some embodiments, application of thermal energy converts a volume of tissue exposed to RE electrical field to undergo irreversible change. In some embodiments, the application of thermal energy decreases at least a voltage or an amplitude or a pulse width or a duration of electroporation electrical pulse to achieve irreversible damage to a volume of tissue. In one embodiment the electroporation electrical pulse is delivered at a peak voltage in a range of 1 kV-6 kV and a pulse width of 0.5 μs-10 μs. In another embodiment the electroporation electrical pulse is delivered at a peak voltage is <1 kV and a pulse width of 0.5 μs-10 μs. In another embodiment the electroporation electrical pulse is delivered at a peak voltage is >1 kV and a pulse width of <0.5 μs. In one embodiment, the application of thermal energy does not alter the tissue impedance by >50% prior to the application of the IRE electrical pulse and hence significantly altering a shape, a size or a volume of the electrical field.

FIG. 10 is a flow chart illustrating an exemplary process of ablation in accordance with some embodiments of the present specification. At step 1002, a catheter is inserted into a patient. The catheter may be inserted in an esophagus, an endotracheal tube, or in any other location within a body where ablation is performed. The catheter comprises at least two lumens—a first lumen for delivering thermal energy such as in the form of heated water vapor, hot water, or a cryogen, and a second lumen for delivering a guidewire. The first lumen is optionally configured with a thermal or heating chamber with electrodes, in fluid communication with or within the lumen, to heat fluid and convert it to hot water or heated vapor. A distal end of the catheter includes at least one positioning element, such as a disc-shaped positioning member, an inflatable balloon member, or any other positioning member. Further one or more ports are located proximate the one or more positioning elements. In one embodiment, multiple ports are located between two positioning elements. The ports are configured to deliver thermal energy from the catheter. Additionally, one or more electrodes for electroporation are positioned proximate each positioning element. In some embodiments, the electrodes are printed on a surface of the positioning elements. In some embodiments, an electrode is also centrally located within each positioning element.

At step 1004, the one or more positioning elements are deployed to a location proximate a target tissue surface for ablation. At step 1006, a combination of thermal energy and electroporation electrical energy is delivered at the target site. The temperature of the targeted tissue is altered by delivery of thermal energy within 30 minutes of applying electroporation electrical field. In some of the embodiments the temperature of the targeted tissue is altered within five minutes of applying the electroporation electrical field. In some of the embodiments the temperature of the targeted tissue is altered within one minute of applying the electroporation electrical field. In some embodiments, the thermal energy is delivered prior to delivering the electroporation pulse. In some other embodiments, the thermal energy is delivered after delivering the electroporation pulse. In yet other embodiments, the thermal energy is delivered concurrently with the application of the electroporation field.

In one embodiment the temperature of the targeted tissue to be ablated with electroporation is raised to greater that 40° C. for more than 1 second. In another embodiment the volume of the tissue with temperature greater than 40° C. is less than volume of the tissue exposed to the IRE electrical field. In yet another embodiment the volume of the tissue with temperature greater than 40° C. is less than volume of the tissue exposed to an RE electrical field, which surrounds the area covered by the electroporation field. In most embodiments the temperature of the targeted issue is raised to greater than 110° C. for less than 1 second by the application of electroporation electrical field.

In another embodiment the temperature of the targeted tissue to be ablated with electroporation is raised to greater than 50° C. for more than 1 second. In another embodiment the volume of the tissue with temperature greater than 50° C. is less than volume of the tissue exposed to the electroporation field. In yet another embodiment the volume of the tissue with temperature greater than 50° C. is less than volume of the tissue exposed to the RE electrical field, which surrounds the area covered by the electroporation field. In most embodiments the temperature of the targeted issue is raised to greater than 110° C. for less than 1 second by the application of electroporation electrical field.

In yet another embodiment the temperature of the targeted tissue to be ablated with electroporation is raised to greater than 60° C. for more than 1 second. In another embodiment the volume of the tissue with temperature greater than 60° C. is less than volume of the tissue exposed to the electrical field. In yet another embodiment the volume of the tissue with temperature greater than 60° C. is less than volume of the tissue exposed to the RE electrical field. In most embodiments the temperature of the targeted issue is raised to greater than 110° C. for less than 1 second by the application of electroporation electrical field.

In yet another embodiment the temperature of the targeted tissue to be ablated with electroporation is raised to greater than 70° C. for more than 1 second. In another embodiment the volume of the tissue with temperature greater than 70° C. is less than volume of the tissue exposed to the electroporation electrical field. In yet another embodiment the volume of the tissue with temperature greater than 70° C. is less than volume of the tissue exposed to the RE electrical field. In most embodiments the temperature of the targeted issue is raised to greater than 110° C. for less than 1 second by the application of electroporation electrical field.

In yet another embodiment the temperature of the targeted tissue to be ablated with electroporation is raised to greater than 80° C. for more than 1 second. In another embodiment the volume of the tissue with temperature greater than 80° C. is less than volume of the tissue exposed to the electroporation electrical field. In yet another embodiment the volume of the tissue with temperature greater than 80° C. is less than volume of the tissue exposed to the RE electrical field. In most embodiments the temperature of the targeted issue is raised to greater than 110° C. for less than 1 second by the application of electroporation electrical field.

In another embodiment the temperature of the targeted tissue to be ablated with electroporation is lowered to less than 25° C. for more than 1 second. In another embodiment the volume of the tissue with temperature less than 25° C. is less than volume of the tissue exposed to the electroporation electrical field. In yet another embodiment the volume of the tissue with temperature less than 25° C. is less than volume of the tissue exposed to the RE electrical field. In most embodiments the temperature of the targeted issue is lowered to a level that is less than 0° C. In most embodiments the temperature of the targeted issue is raised to greater than 110° C. for less than 1 second by the application of electroporation electrical field.

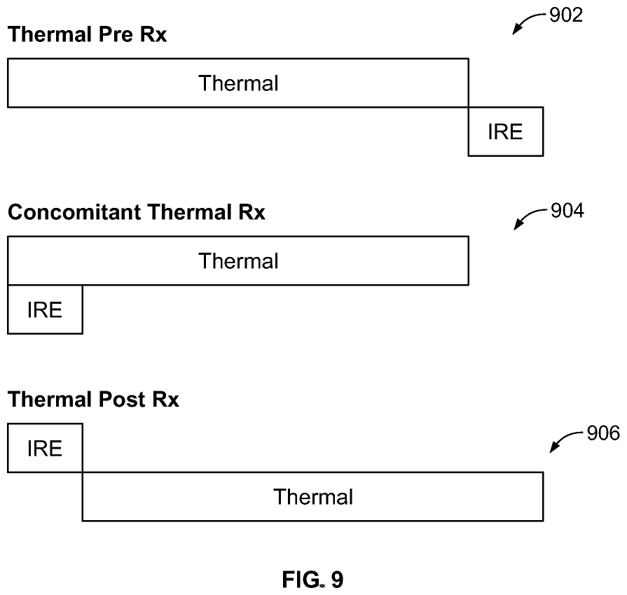

FIG. 11 is a flow chart illustrating another exemplary process of ablation in accordance with an embodiment of the present specification. At step 1102, a catheter is inserted into a patient. The catheter may be inserted in an esophagus, an endotracheal tube, or in any other location within a body where ablation is performed. The catheter comprises at least two lumens—a first lumen for delivering thermal energy such as in the form of heated water vapor, hot water, or a cryogen, and a second lumen for delivering a guidewire. The first lumen is optionally configured with a thermal or heating chamber with electrodes, in fluid communication with or within the lumen, to heat fluid and convert it to hot water or heated water vapor. A distal end of the catheter includes at least one positioning element, such as a disc-shaped positioning member, an inflatable balloon member, or any other positioning member. Further one or more ports are located proximate the one or more positioning elements. In one embodiment, multiple ports are located between two positioning elements. The ports are configured to deliver thermal energy from the catheter. Additionally, one or more electrodes for electroporation are positioned proximate each positioning element. In some embodiments, the electrodes are printed on a surface of the positioning elements. In some embodiments, an electrode is also centrally located within each positioning element.

At step 1104, the one or more positioning elements are deployed to a location proximate a target tissue surface for ablation. At step 1106, thermal energy is applied to raise the target tissue temperature to a range within 40° C. and 100° C. for a duration of greater than 1 second. At step 1108, electrical energy is delivered for electroporation to raise the target tissue temperature to greater than 100° C. for a duration of less than 1 second. In some embodiments, a temperature of the target tissue area is lowered to less than 25° C. but greater than −200° C. for more than 1 second.

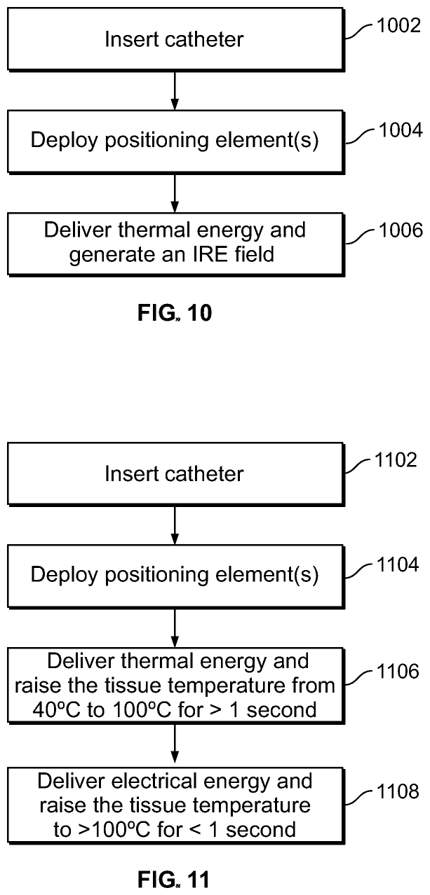

FIG. 12 is a flow chart illustrating another exemplary process of ablation in accordance with an embodiment of the present specification. At step 1202, a catheter is inserted into a patient. The catheter may be inserted in an esophagus, an endotracheal tube, or in any other location within a body where ablation is performed. The catheter comprises at least two lumens—a first lumen for delivering thermal energy such as in the form of heated water vapor, hot water, or a cryogen, and a second lumen for delivering a guidewire. The first lumen is optionally configured with a thermal or heating chamber with electrodes, in fluid communication with or within the lumen, to heat fluid and convert it to hot water or heated vapor. A distal end of the catheter includes at least one positioning element, such as a disc-shaped positioning member, an inflatable balloon member, or any other positioning member. Further one or more ports are located proximate the one or more positioning elements. In one embodiment, multiple ports are located between two positioning elements. The ports are configured to deliver thermal energy from the catheter. Additionally, one or more electrodes for electroporation are positioned proximate each positioning element. In some embodiments, the electrodes are printed on a surface of the positioning elements. In some embodiments, an electrode is also centrally located within each positioning element.

At step 1204, the one or more positioning elements are deployed to a location proximate a target tissue surface for ablation. At step 1206, thermal energy is applied to lower the target tissue temperature to a value that is less than 25° C. for a duration of greater than 1 second. At step 1208, electrical energy is delivered for electroporation to raise the target tissue temperature to greater than 100° C. for a duration of less than 1 second.

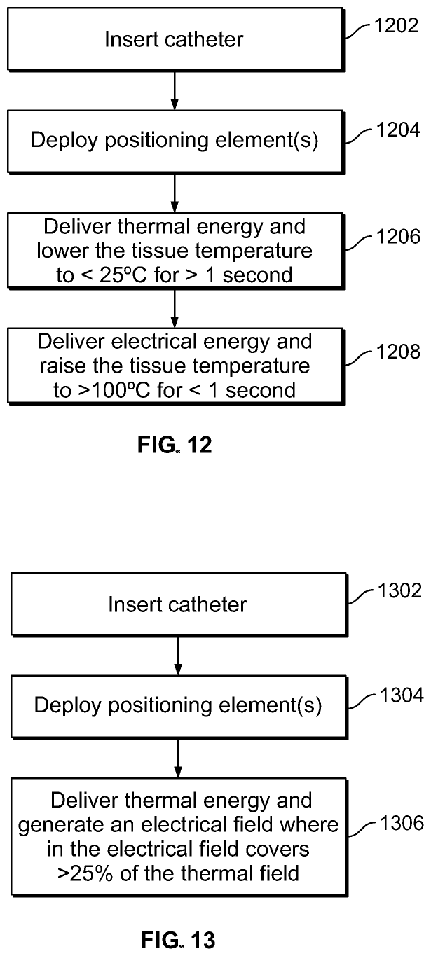

FIG. 13 is a flow chart illustrating still another exemplary process of ablation in accordance with an embodiment of the present specification. At step 1302, a catheter is inserted into a patient. The catheter may be inserted in an esophagus, an endotracheal tube, or in any other location within a body where ablation is performed. The catheter comprises at least two lumens—a first lumen for delivering thermal energy such as in the form of heated water vapor, and a second lumen for delivering a guidewire. The first lumen is optionally configured with a thermal or heating chamber with electrodes, in fluid communication with or within the lumen, to heat fluid and convert it to hot water or heated vapor. A distal end of the catheter includes at least one positioning element, such as a disc-shaped positioning member, an inflatable balloon member, or any other positioning member. Further one or more ports are located proximate the one or more positioning elements. In one embodiment, multiple ports are located between two positioning elements. The ports are configured to deliver thermal energy from the catheter. Additionally, one or more electrodes for electroporation are positioned proximate each positioning element. In some embodiments, the electrodes are printed on a surface of the positioning elements. In some embodiments, an electrode is also centrally located within each positioning element.

At step 1304, the one or more positioning elements are deployed to a location proximate a target tissue surface for ablation. At step 1306, a combination of thermal energy and electroporation electrical energy is delivered at the target site such that an electrical field generated by the electrical energy covers more than 25% of the thermal field generated by the thermal energy.

There are several advantages of applying thermal energy with electroporation energy. Application of thermal energy decreases at least one of the capacitor size, peak voltage, or pulse width requirement of an electroporation treatment. Application of controlled thermal energy within 30 minutes of applying electroporation energy results in thermal potentiation of the electroporation effect and increases the depth or the volume of the electroporation lesion. In some cases, pretreatment with a controlled thermal energy delivery to a target tissue within 15 minutes, and more specifically within five minutes of delivering electroporation treatment improves the effect of electroporation, with the best effect being when the thermal energy is delivered less than 1 minute prior to the application of electroporation as the onset of edema from the thermal injury increase the thickness of the tissue and decreases both the reversible and irreversible electroporation fields. Applying the thermal energy after application of electroporation pulses do not suffer from the limitation caused by tissue edema. Thermal energy is typically applied between 40° C. and 110° C. for a duration between 1 seconds and 30 minutes without raising the tissue pressure above 5 atm. The electroporation energy is applied such that the temperature of the targeted issue is raised to greater than 110° C. for less than 1 second. Concomitant application of thermal energy and electroporation energy results in potentiation of both the thermal effect and electroporation effect resulting in a synergistic effect with the two modalities.

In some embodiments, electrical energy is applied to the tissue for a duration less than 1 seconds.

In some embodiments, a thermal energy is applied to raise the temperature of the target tissue area is to greater that 40° C. but less than 100° C. for more than 1 second and an electrical energy field is applied to raise the temperature of the target tissue area to greater than 100° C. for less than 1 second.

In some embodiments, a thermal energy is applied to lower the temperature of the target tissue area is to less than 25° C. for more than 1 second and an electrical energy field is applied to raise the temperature of the target tissue area to greater than 100° C. for less than 1 second.

In some embodiments, a thermal energy is applied to raise the temperature of the target tissue area to greater that 40° C. but less than 100° C. for more than 1 second and a reversible electroporation electrical field is applied for less than 1 second to cause an irreversible tissue change.

In some embodiments, a thermal energy is applied to lower the temperature of the target tissue area to less than 25° C. for more than 1 second and a reversible electroporation electrical field is applied for less than 1 second to cause an irreversible tissue change.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for tissue ablation, comprising:
a catheter with a proximal end, a distal end, and a lumen, comprising:
a thermal chamber in fluid communication with the lumen;
at least one first positioning element positioned proximate the distal end of the catheter;
at least one delivery port positioned proximate the distal end of the catheter and configured to deliver thermal energy generated within the thermal chamber; and
at least one electrode positioned proximate the distal end of the catheter and configured to generate an electrical field to cause electroporation when activated; and
a controller comprising a microprocessor for controlling the delivery of the thermal energy and the generating of the electrical field.

2. The system of claim 1, wherein the thermal chamber is positioned within the lumen.

3. The system of claim 1, wherein a size of a thermal field created by thermal energy is a different size than the electrical field.

4. The system of claim 1, wherein the at least one first positioning element comprises any one of a wire mesh, disc, hood, cap, or inflatable balloon.

5. The system of claim 1, wherein the at least one first positioning element comprises any one of a circular, oval, rectangular, conical, spherical, oblong, or square shape.

6. The system of claim 1, further comprising at least one needle at the distal end of the catheter wherein the at least one needle includes the at least one delivery port.

7. The system of claim 1, wherein the at least one electrode configured to generate an electrical field is positioned on the at least one first positioning element.

8. The system of claim 7, wherein the at least one electrode configured to generate an electrical field is printed on a surface of the at least one first positioning element.

9. The system of claim 1, further comprising at least one second positioning element proximate a distal end of catheter.

10. The system of claim 9, wherein the at least one second positioning element comprises any one of a wire mesh, disc, hood, cap, or inflatable balloon.

11. The system of claim 9, wherein the at least one first positioning element comprises any one of a circular, oval, rectangular, conical, spherical, oblong, or square shape.

12. The system of claim 9, wherein the at least one delivery port configured to deliver thermal energy is positioned on the catheter between the at least one first positioning element and the at least one second positioning element.

13. A method for ablation of a target tissue area, comprising:
inserting a catheter comprising at its distal end at least one positioning element, at least one port proximate the at least one positioning element configured to deliver thermal energy, and at least one electrode proximate the at least one positioning element configured to generate an electric field to cause electroporation;
deploying the distal end proximate the target tissue area;
applying a combination of thermal energy from the at least one port and an electrical field from the at least one electrode.

14. The method of claim 13, wherein thermal energy is applied before applying the electrical field.

15. The method of claim 13, wherein thermal energy is applied at the same time that the electrical field is applied.

16. The method of claim 13, wherein thermal energy is applied after applying the electrical field.

17. The method of claim 13, wherein a temperature of the target tissue area is raised to greater than 40° C. for more than 1 second.

18. The method of claim 13, wherein a temperature of the target tissue area is raised to greater than 40° C. and less than 100° C. for more than 1 second.

19. The method of claim 13, wherein a temperature of the target tissue area is lowered to less than 25° C. and greater than −200° C. for more than 1 second.

20. The method of claim 13, wherein a temperature of the target tissue area is raised to greater than 100° C. for less than 1 second.

21. The method of claim 13, wherein a temperature of the target issue area is raised to greater than 110° C. for less than 1 second.

22. The method of claim 13, wherein thermal energy is applied for a duration between 1 second and 30 minutes.

23. The method of claim 13, wherein electrical energy is applied for a duration of less than 1 second.

24. The method of claim 13, wherein a pressure at the target tissue area is not increased above 5 atm.

25. The method of claim 13, wherein a temperature of the target tissue area is altered within five minutes of applying the electrical field.

26. The method of claim 13, wherein the thermal energy is applied to raise a temperature of the target tissue area to greater than 40° C. and less than 100° C. for more than 1 second, and the electric field is applied to raise a temperature of the target tissue area to greater than 100° C. for less than 1 second.

27. The method of claim 13, wherein the thermal energy is applied to lower a temperature of the target tissue area to less than 25° C. for more than 1 second, and the electric field is applied to raise a temperature of the target tissue area to greater than 100° C. for less than 1 second.

28. The method of claim 13, wherein the thermal energy is applied to raise a temperature of the target tissue area to greater than 40° C. and less than 100° C. for more than 1 second, and the electric field is applied for electroporation for less than 1 second to cause an irreversible tissue change.

29. The method of claim 13, wherein the thermal energy is applied to lower a temperature of the target tissue area to less than 25° C. for more than 1 second, and the electric field is applied for electroporation for less than 1 second to cause an irreversible tissue change.
